(19) United States
(12) Patent Application Publication
(10) Pub. No.: US 2004/0230219 A1
(43) Pub. Date: Nov. 18, 2004

(54) Anchoring, supporting and centering catheter system for treating chronic total occlusions

(76) Inventor: Leo R. Roucher JR., Rancho Santa Fe, CA (US)

Correspondence Address: MICHAEL E. KLICPERA, PO BOX 573, LA JOLLA, CA, 92038-0573, US

(21) Appl. No.: 10/435,903

(22) Filed: May 12, 2003

(51) Int. Cl. A61M 29/00

(52) U.S. Cl. 606/194

(57) ABSTRACT

A system and associated methods provided for crossing total occlusions in blood vessels. While the system and methods are particularly beneficial for the treatment of coronary artery disease, they are also useful in the treatment of other arteries and veins, such as the treatment of peripheral vascular diseases. The present invention used a system comprised of three unique and specialized components: 1. an anchoring, supporting and centering balloon sheath apparatus; 2. a hydraulic guidewire with removable core; and 3. an exchange sheath.

### FIELD OF THE INVENTION

The present invention relates generally to medical devices and their associated methods of use. More particularly, the present invention relates to an anchoring, centering, and supporting catheter system and associated procedures for crossing chronic total occlusions in blood vessels using a standard or specialized guidewire.

### BACKGROUND OF THE INVENTION

Cardiovascular disease is commonly accepted as being one of the most serious health risks facing our society today. Diseased and obstructed coronary arteries can restrict the flow of blood and cause tissue ischemia and necrosis. After over two decades of investigation, although the exact etiology of sclerotic cardiovascular disease is still in question, the treatment of narrowed coronary arteries is more defined. Surgical construction of coronary artery bypass grafts (CABG) is often the method of choice when there are several diseased segments in one or multiple arteries. Open heart surgery is, of course, very traumatic for patients. In many cases, less traumatic, alternative methods are available for treating cardiovascular disease percutaneously. These alternate treatment methods generally employ various types of percutaneous transluminal angioplasty (PTCA) balloons or excising devices (atherectomy) to remodel or debulk diseased vessel segments. A further alternative treatment method involves percutaneous, intraluminal installation of expandable, tubular stents or prostheses in sclerotic lesions.

A particularly troublesome form of cardiovascular disease results when a blood vessel becomes totally occluded with atheroma or plaque, referred to as a chronic total occlusion. Until recently, chronic total occlusions have usually been treated by performing a bypass procedure where an autologous or synthetic blood vessel is anastomotically attached to locations on the blood vessel upstream and downstream of the occlusion. While highly effective, such bypass procedures are quite traumatic to the patient.

Medical catheters such as balloon catheters have been proven efficacious in treating a wide variety of blood vessel disorders. Moreover, these types of catheters have permitted clinicians to treat disorders with minimally invasive procedures that, in the past, would have required complex and perhaps life-threatening surgeries. For example, balloon angioplasty is now a common procedure to alleviate stenotic lesions (i.e., clogged arteries) in blood vessels, thereby reducing the need for heart bypass operations.

Because medical catheters and guidewires must be passed through a tortuous blood vessel network to reach the intended treatment site, it is desirable that the catheter be fairly flexible, especially at the distal end. However, the distal end must not be so flexible that it tends to bend back upon itself (prolaspe) when the clinician advances the catheters or guidewires distal end through the tortuous vasculature.

Recently, catheter-based intravascular procedures have been utilized to treat chronic total occlusions with limited success. Catheter-based intravascular procedures include angioplasty, atherectomy, stenting, and the like, and are often preferred over invasive coronary artery bypass graft (CABG) procedures because they are much less traumatic to the patient. In some instances, before an interventional catheter treatment can be performed, it is sometimes necessary to cross a significant occlusion with a guidewire to provide access for the interventional catheter. However, in many cases, the guidewire inadvertently penetrates into the subintimal space between the intimal layer and the adventitial layer of the blood vessel, or worse yet, perforates the arterial wall, as it attempts to cross the occlusion. Once in the subintimal space or perforated, it is very difficult and in many cases impossible to direct the guidewire back into the blood vessel lumen. In such cases, it will usually be impossible to perform the interventional procedure and other, more traumatic, surgical procedures may have to be employed.

For these reasons, it would be desirable to provide methods and a system that facilitates crossing a chronic total occlusion in a blood vessel. In particular, it would be desirable to provide an anchoring, centering, and supporting catheter system which could be used with a specialized guidewire to direct the guidewire away from the subintimal space and into the occluded blood vessel lumen. Such methods and apparatus should be useful in coronary arteries as well as peripheral and other blood vessels and should be capable of being performed with or without imaging from within or adjacent to the blood vessel.

### SUMMARY OF THE INVENTION

According to the present invention, a system and methods are provided for crossing total occlusions in blood vessels. While the system and methods are particularly beneficial for the treatment of coronary artery disease, they are also useful in the treatment of other arteries and veins, such as the treatment of peripheral vascular diseases.

The present invention uses a system comprised of three unique and specialized components:

1. An Anchoring, Supporting and Centering Balloon Sheath Apparatus;

2. A Hydraulic Guidewire with Removable Core; and

3. An Exchange Sheath.

The total occlusions are crossed by not forming a track into the subintimal space between the intimal layer and the adventitial layer of a blood vessel, but rather the present invention functions as an anchoring, centering, and supporting catheter system and associated procedures for crossing chronic total occlusions without penetrating the subintimal space. In another embodiment of the present invention, one or more directing wires are provided which present additional control of the general heading or bearing of the guidewire.

The track of the guidewire is generally directed towards the original luminal space from a location proximal to the total occlusion to a location that is distal to the total occlusion. The Balloon Sheath functions to facilitate the centering of the guidewire into the total occlusion. Hydraulic means is provided which is directed from the tip of the guidewire to provide additional centering through the total occlusion.

By continuing to advance the wire, it will usually pass substantially through the total occlusion of the original luminal space and can be further advanced to the desired distal location. Now the Exchange Sheath is advanced over the guidewire to further dilate and dotter the channel established by the guidewire. Once the Exchange Sheath is passed through the total occlusion, the hydraulic guidewire or other guidewire can be used to introduce interventional balloons, stents or other devices to achieve a clinically desirable patent vessel lumen.

In typical methods, the guidewire is directed using an anchoring, centering and supporting balloon catheter. Generally, the balloon catheter is advanced just proximal of the total occlusion with the guidewire advanced to a distal position in the catheter guidewire lumen. A flexible balloon on the distal end of the catheter is inflated to a working pressure whereby the guidewire distal lumen is directed substantially towards the original luminal space. The guidewire and the catheter are then manipulated so that the wire is deflected substantially towards the center of the original lumen. The balloon catheter is also useful in supporting the wire as it is advanced into and/or through the track, i.e. the catheter can enhance the “pushability” of the wire when it is advanced forward through the resisting material. Hydraulic means are provided in the guidewire to perform two functions. Side directed hydraulic means function as thrusters which facilitate the centering of the guidewire through the total occlusion. Forward directed hydraulic means functions to assist pushability of the guidewire through the occlusion.

It will usually be necessary to determine when the guidewire and/or balloon catheter are positioned distal to the total occlusion so that the wire may be directed towards the center of the blood vessel lumen and beyond said occlusion. Most simply, such position determination can be made by fluoroscopically imaging the blood vessel in a conventional manner. Alternatively or additionally to such fluoroscopic imaging, intravascular imaging, e.g. intravascular ultrasonic imaging (IVUS), and a variety of optical imaging modalities, such as optical coherence tomography (OCT), may be employed.

After the passage or channel is formed through the occluded blood vessel lumen and the hydraulic wire is in place across the total occlusion, the Exchange Sheath is advanced through the passage or channel to further dilate and dotter the channel. After the Exchange Sheath is advanced through the occlusion, it can be retracted, or the guidewire can be exchanged and then the Exchange Sheath removed. Then either the hydraulic guidewire, a previously placed standard guidewire, or a new guidewire can be available for use in positioning interventional and diagnostic catheters across the total occlusion. Most commonly, interventional catheters will be positioned across the total occlusion for treating the occlusion. Exemplary interventional catheters include angioplasty balloon catheters, rotational atherectomy catheters, directional atherectomy catheters, stent-placement catheters, and the like.

### BRIEF DESCRIPTION OF THE DRAWINGS

FIG. 1 is an illustration of a typical environment that the anchoring, supporting and centering balloon sheath, hydraulic guidewire and sheath apparatus system.

FIG. 2 is a cross-sectional illustration of a distal end of the balloon sheath embodiment with the balloon in a deflated configuration.

FIG. 3 is a cross sectional illustration of a distal end of the balloon sheath embodiment that employs control wires engaged to the proximal side of the balloon with the balloon in an expanded configuration.

FIG. 4 is a cross-sectional illustration of a distal end of the balloon sheath embodiment that employs control wires engaged to the distal side of the balloon with the balloon in an expanded configuration.

FIG. 5 is a cross-sectional view taken along the line 5-5 of FIG. 2.

FIG. 6 is a cross-sectional view taken along the line 6-6 of FIG. 2.

FIG. 7 is a cross-sectional view taken along the line 7-7 of FIG. 3 and line 7-7 of FIG. 4.

FIG. 8 is a cross-sectional illustration of a hydraulic guidewire with an attached adapter with at least one extension mounted on said proximal end of said guidewire.

FIG. 9 is a cross-sectional view taken along the line 9-9 of FIG. 8.

FIG. 10 is a cross-sectional illustration of an example of the exchange sheath of the present invention system.

FIG. 11 is a cross-sectional view taken along the line 11-11 of FIG. 10.

FIG. 12 is a cross-sectional view of a prior standard guidewire penetrating the vessel wall.

FIG. 13 is a cross-sectional view of a prior standard guidewire non-centered into the vessel lumen and entering and advancing into the subintimal space within the medial layer.

FIG. 14 is a cross-sectional view of a prior standard guidewire prolapsing within a vessel lumen.

FIG. 15 is a cross-sectional view of the present invention hydraulic guidewire in position just proximal of the total occlusion for treatment.

FIG. 16 is a cross-sectional view of the present invention balloon sheath in an expanded configuration with the hydraulic guidewire just proximal to the total occlusion.

FIG. 17 is a cross-sectional view of the present invention balloon sheath in an expanded configuration with the hydraulic guidewire employing various apertures to further center the guidewire and penetrate the total occlusion.

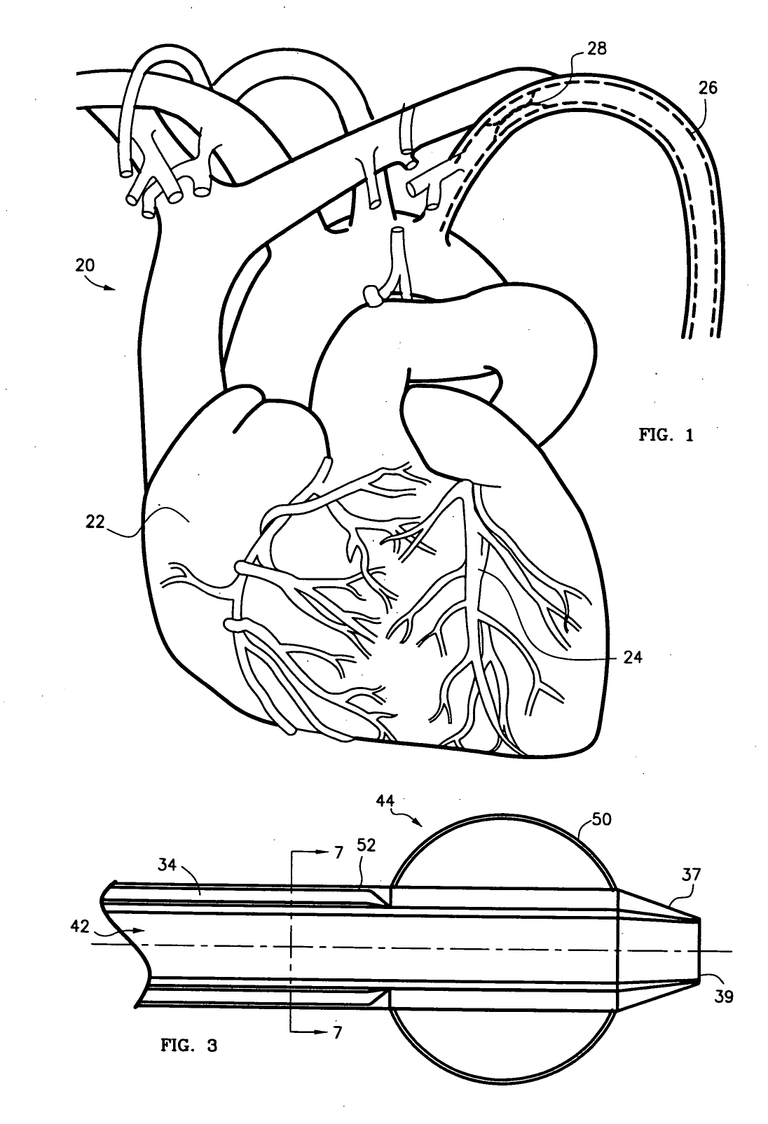

### DESCRIPTION OF THE INVENTION

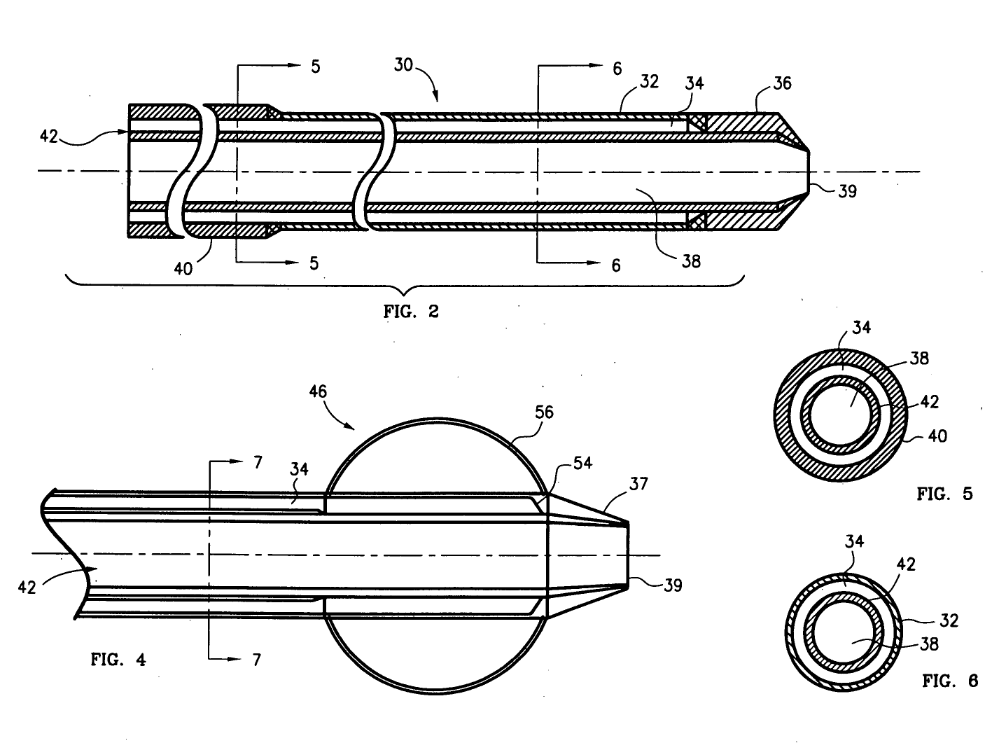

With reference now to the exemplary drawings, and particularly to FIGS. 2, 3 and 4 there are shown three cross-sectional illustrations of a distal end of the balloon sheath embodiment with the balloon in a deflated configuration in accordance with the present invention system. FIG. 2 shows a first embodiment 30 of the balloon sheath embodiment with the balloon in a deflated configuration. This FIG. 2 demonstrates that the catheter has a proximal catheter shaft 42 and a distal catheter shaft 32 both of a coaxial design with an inflation lumen 34 and a guidewire lumen 38. Closer to the distal end the shaft 32 has a slightly reduced diameter with increased flexibly characteristics. The catheter shafts 40 and 32 are typically fabricated from a mixture of low density (LDPE) and high density (HDPE) polyethylenes, or can be manufactured from a variety of thermoplastic polymers know by those skilled in the art in making medical catheters. The distal section of the catheter is fitted with a flexible balloon 36 comprised of a C-flex or latex material that, rather than designed to apply dilatation force to a vessel wall, expands under relatively low working pressures to a substantially spherical balloon configuration. The general characteristic of this catheter assembly is that the flexible (expanded spherical balloon 50,56 conforms to the contour of the vessel wall, anchoring the catheter in place. In this position, the balloon 36 now provides support to facilitate centering of an advancing hydraulic guidewire into the lumen of the vessel and through the total occlusion. The present invention system flexible balloon 36 is expanded with relatively low working pressures ranging from 0.5 to 4 atmospheres (atm.), and preferably from 1 to 2 atms. Extending throughout the length of the catheter is a guidewire lumen 38 having dimensional specifications to receive the present invention system hydraulic guidewire. Typically, the guidewire lumen is 0.012″ to 0.020″ in diameter, and preferably 0.014″ to 0.018″. The catheter shaft terminates in a distal opening 39 whereby the hydraulic guidewire protrudes forward from the catheter. Not shown at the proximal end of the catheter is a typical two or three armed adapter which design, materials of construction and means for attaching to the catheter shaft are well know by those skilled in the art.

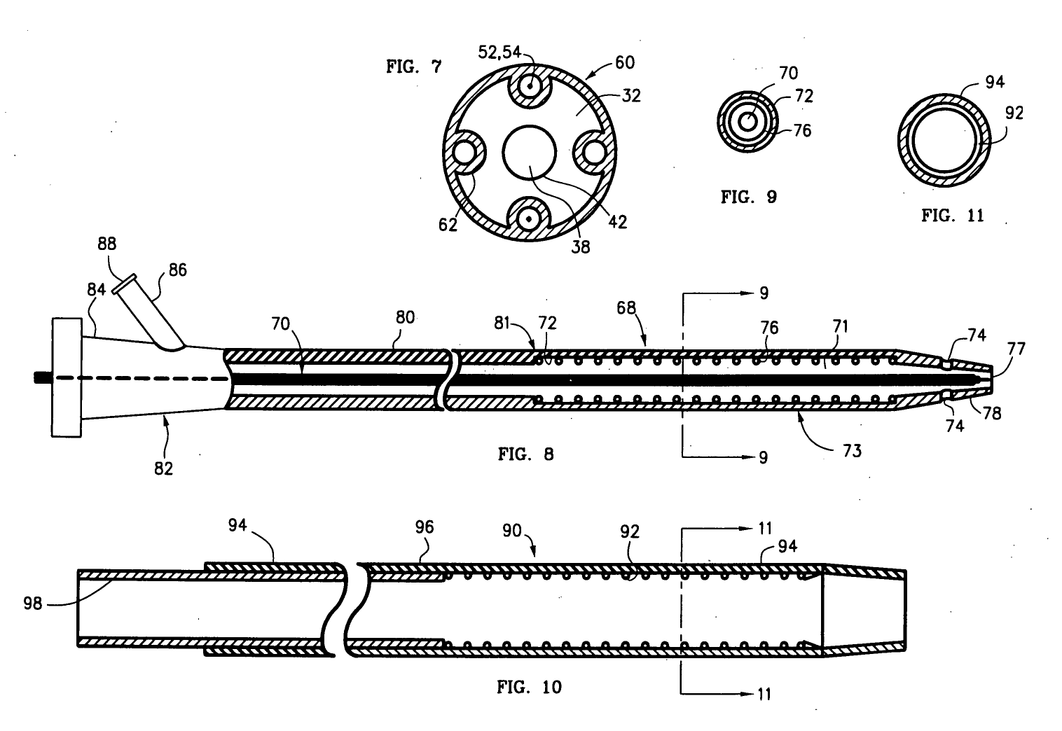

FIG. 3 is a cross sectional illustration of a distal end of the balloon sheath embodiment which employs control wires engaged to the proximal side of the balloon with the balloon in an expanded configuration. Shown on FIG. 3 is a second embodiment 44 of the balloon sheath whereby the catheter/spherical balloon assembly employs a means to attach one or more control wires 52 to the proximal end of the balloon. The proximally attached control wires 52 extend along the length of the catheter and are contained with channels 62 shown in FIG. 7. The channels 62 terminate at the proximal end of the flexible balloon 50. The proximally attached control wires 52 engage the end of the channel and are affixed using an adhesive, shrink heat technology, or employing an enlarged (e.g. ball) configuration at the distal end of the control wire that inhibits the distal end from entering the channel 62. The catheter shafts are typically fabricated from a mixture of low density (LDPE) and high density (HDPE) polyethylenes, or can be manufactured from a variety of thermoplastic polymers know by those skilled in the art in making medical catheters. The flexible balloon catheter and the guidewire lumen are dimensionally and characteristically similar with that discussed previously for the first embodiment.

FIG. 4 is a cross-sectional illustration of a distal end of the balloon sheath embodiment that employs control wires engaged to the distal side of the balloon with the balloon in an expanded configuration. Shown on FIG. 4 is a third embodiment 46 of the balloon sheath whereby the catheter/spherical balloon assembly employs a means to attach one or more control wires 54 to the distal end of the balloon. The distally attached control wires 54 extend along the length of the catheter and are contained with channels 62 shown in FIG. 7. The channels 62 terminate at the distal end of the flexible balloon 56. The distally attached control wires 54 engage the end of the channel and are affixed using an adhesive, shrink heat technology, or employing an enlarged (e.g. ball) configuration at the distal end of the control wire that inhibits the distal end from entering the channel 62. The catheter shafts are typically fabricated from a mixture of low density (LDPE) and high density (HDPE) polyethylenes, or can be manufactured from a variety of thermoplastic polymers know by those skilled in the art in making medical catheters. The flexible balloon catheter and the guidewire lumen are dimensionally and characteristically similar with that discussed previously for the first embodiment.

FIG. 5 is a cross-sectional view taken along the line 5-5 of FIG. 2 and shows the coaxial design and relative positioning of the proximal catheter shaft, demonstrating the outer shaft tubing material 40, the inflation/deflation lumen 34, inner shaft tubing material 42 and guidewire lumen 38.

Likewise, FIG. 6 is a cross-sectional view taken along the line 6-6 of FIG. 2 and shows the coaxial design and relative positioning of the distal catheter shaft, demonstrating the outer shaft tubing material 32, the inflation/deflation lumen 34, inner shaft tubing material 42 and guidewire lumen 38.

FIG. 7 is a cross-sectional view taken along the line 7-7 of FIG. 3 and line 7-7 of FIG. 4 and demonstrates the multiple lumen tubing 60 that includes channels 62 for containing the embodiments having control wires 52, 54 for engaging the flexible balloon. This Figure shows four channels located at ninety degrees. It is contemplated by the Applicants that one to six wires may be employed to facilitate pivotal movement of the flexible balloon. In its simplest form, a control wire is pulled away from the armed adapter on the proximal end of the catheter in a direction that is substantially parallel to the longitudinal axis. The tensioned control wire that is attached to the balloon will compel the balloon to pivot and rotate in the direction of the tensioned control wire. If the control wire located in the 12 o'clock position of FIG. 7 is tensioned, the balloon will tend to bend towards this 12 o'clock position. The lumen of channel 62 is generally 0.002″ to 0.010″ in diameter, with a preferable range of 0.003″ to 0.005″. The control wires 52, 54 are generally smaller than the lumen of the channel by approximately 0.002″, therefore, the control wire 52, 54 is generally 0.001″ to 0.008″ in diameter, with a preferable range of 0.001″ to 0.003″. Control wires must have sufficient tensional (pull) strength to not break when subjected to a range of tensional forces. Therefore, the control wires 52, 54 are fabricated from a metallic material such as stainless steel. Other metallic and polymeric materials with adequate tensional (pull) strength could suffice for the control wire material. It is also contemplated by the Applicants, but not shown, that the channels and corresponding control wires could be configured in another shape, such as square or rectangle.

Now referring to FIG. 8 which is a cross-sectional illustration of the hydraulic guidewire 68 of the present invention system. The hydraulic guidewire 68 includes a proximal stainless steel hypotube structure 80 that terminates at approximately the last 35 centimeters as a spring coil 76 encased with a polymeric jacket 72 to form a composite structure 73. Attached to the proximal end of the guidewire is an adapter with at least one extension. In FIG. 8, the two arm adapter has a hydraulic fitting 86 with a hydraulic lumen 88 and a removable core fitting 84. One type of spring coil 76 can be a 0.002″×0.004″ rectangle fabricated from a spring stainless steel. The polymeric jacket is typically a Pebax material that is shrink fitted over the spring coil 76. Other polymeric materials, such as Teflon, HDPE/LDPE, or urethanes can be employed. The junction 81 between the hypotube 80 and the composite structure 73 is secured by techniques known in the prior art, such as over lapping one section over another and adhering by brazing or adhesives. The lumen of the hydraulic guidewire is generally 0.006″ to 0.010″ in diameter, preferably with a range of 0.008″ to 0.010″ in diameter. The end of the guidewire is tapered and terminates with an aperture 77 that is substantially aligned with the longitudinal axis of the hydraulic guidewire. Fluid (saline, contrast) infused from the proximal hydraulic fitting 86 can be a steady stream or pulsed through the lumen 71 with sufficient pressure to exit this tapered end 78 and out the aperture 77. Together with mechanical force, the pulsed liquid will assist the guidewire in penetrating the total occlusion.

Furthermore, the hydraulic guidewire 68 can be fitted with one or more apertures 74 facing radially from the longitudinal axis of the guidewire. The apertures 74 will perform as thrusters assisting the guidewire to center within a vessel lumen. When fluid is infused from the proximal hydraulic fitting 86 with sufficient pressure through the lumen 71 to exit one or more of these apertures 74, they can serve to facilitate centering the guidewire.

The hydraulic guidewire 68 also includes a removable core 70 that is used initially to provide internal support to the guidewire 68. The removable core 70 can be relocated at various points along the distal end to vary the stiffness and flexibility for given clinical requirements. Furthermore the removable core 70 can be completely retracted when hydraulic fluids are being infused through the guidewire lumen 71.

FIG. 9 is a cross-sectional view taken along the line 9-9 of FIG. 8 and shows the design and relative positioning of the removable core 70, the spring coil 76 and the guidewire jacket 72.

FIG. 10 is a cross-sectional illustration of the exchange sheath of the present invention system. The exchange sheath 90 includes a spring coil 92 encased with a polymeric jacket 94 to form an exchange sheath composite structure 95. One type of spring coil 76 can be a 0.002″×0.004″ rectangle fabricated from a spring stainless steel. The polymeric jacket is typically a Pebax material that is shrink fitted over the spring coil 76. Other polymeric materials, such as Teflon, HDPE/LDPE, or urethanes can be employed. The lumen of the exchange sheath is generally 0.018″ to 0.026″ in diameter, preferably with a range of 0.020″ to 0.022″ in diameter. The end of the exchange sheath is tapered. The exchange sheath 90 is designed to advance over the hydraulic guidewire 68, penetrating the guidewire-crossed total occlusion, further dilating and dottering the channel or lumen first created by the hydraulic guidewire. After the exchange sheath is advanced through the occlusion, it can be retracted, or the guidewire can be exchanged and then the exchange sheath removed. Then either the hydraulic guidewire, a previously placed standard guidewire, or a new guidewire can be available for use in positioning interventional and diagnostic catheters across the total occlusion. Most commonly, interventional catheters will be positioned across the total occlusion for treating the occlusion. Exemplary interventional catheters include angioplasty balloon catheters, rotational atherectomy catheters, directional atherectomy catheters, stent-placement catheters, and the like.

FIG. 11 is a cross-sectional view taken along the line 11-11 of FIG. 10 and shows the design and relative positioning of the spring coil 92 and the exchange sheath jacket 94.

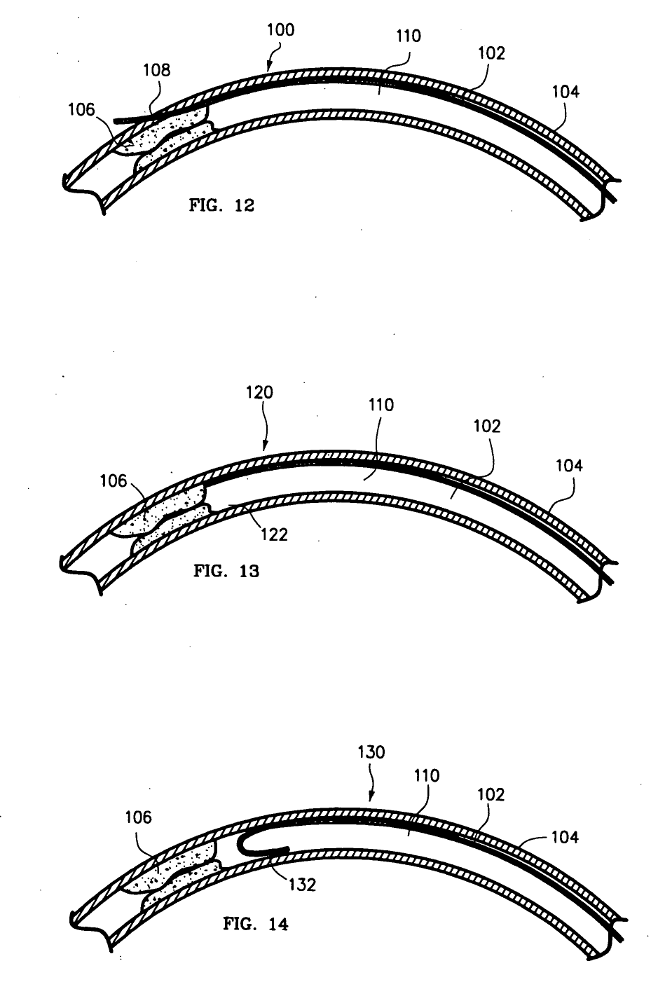

FIG. 12 is a cross-sectional view of a prior standard guidewire where the guidewire has entered the subintimal space and penetrated the vessel wall 104. The medical equipment currently in use has a fundamental flaw because it tends to track the outside radius of the vessel lumen 110. Once the guidewire has penetrated the vessel wall 108, it is very difficult to retract the guidewire and direct it towards and through the total occlusion 106. Furthermore, the penetrated vessel wall may need further interventions to minimize blood perfusion.

FIG. 13 is a cross-sectional view of a prior standard guidewire non-centered into the vessel lumen and entering and advancing into the subintimal space within the medial layer. Medical equipment currently in use has a fundamental flaw in that it tends to track the outside radius of the vessel lumen 110. Once the guidewire 122 has entered the subintimal space of the vessel wall 104, it is very difficult to retract the guidewire and direct it towards and through the total occlusion 106. This result is very undesirable and potentially dangerous to the clinical outcome.

FIG. 14 is a cross-sectional view of a prior standard guidewire prolapsing 132 within a vessel lumen. Even when current technology medical equipment is shaped to facilitate finding the center of the total occlusion, the guidewire can get caught in thrombus or buckle against the occlusion. Further attempts to advance the guidewire may cause the tip to curl over and prolapse over itself. Again, this result is very undesirable and potentially dangerous because it can result in perforation of the artery.

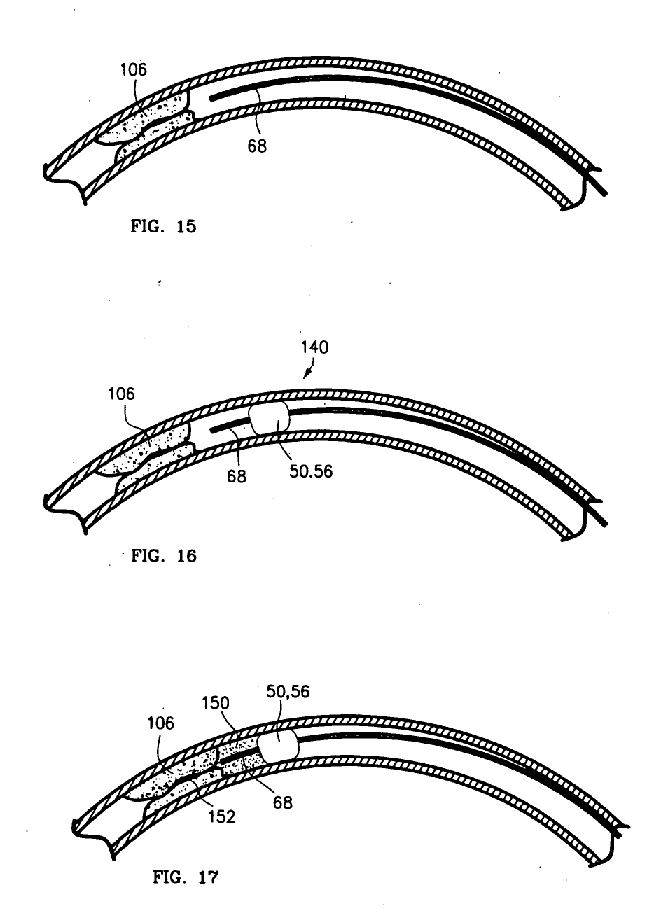

FIG. 15 is a cross-sectional view of the present invention hydraulic guidewire 68 within a vessel lumen and in position just proximal of the total occlusion 106 for treatment.

FIG. 16 is a cross-sectional view of the present invention balloon sheath in an expanded configuration 50, 56 with the hydraulic guidewire 68 just proximal to the total occlusion. The balloon sheath is supporting the guidewire 68 to become substantially centered within the vessel lumen. Furthermore, the balloon sheath with proximally attached control wires 44 or balloon sheath with distally attached control wires 46 can be controlled remotely from the proximal end of the catheter to further enhance the centering position of the guidewire.

FIG. 17 is a cross-sectional view of the present invention balloon sheath in an expanded configuration 50, 56 with the hydraulic guidewire 68 employing various apertures 74 to further center the guidewire and penetrate the total occlusion 106. As can be seen by the present invention system and methods, the balloon sheath and hydraulic guidewire 68 are designed to center the guidewire within the vessel lumen and facilitate the penetration through the total occlusion 106. Hydraulic pulses 152 emanating from the distal tip are assisting the guidewire 68 to penetrate the occlusion 106 and create a channel. Fluids being forced through the radially directed apertures 74 are further causing the hydraulic guidewire 68 to be centered with the vessel lumen. Once the hydraulic guidewire 68 has passed through the total occlusion and created a channel, the balloon sheath can be removed and the exchange sheath can be threaded over the hydraulic guidewire to further dilate and dotter the channel. Access is now available for other interventional devices to further treat the occlusion and achieve a clinically desirably result.

What is claimed is:

1. An anchoring, centering, and supporting system for treating a chronic total occlusions comprising:
a catheter body having a proximal end, a distal end, and at least one lumen extending throughout the length of said catheter body, one lumen including a distal opening and a proximal opening; said catheter having a flexible balloon mounted on the distal end;
a hydraulic guidewire said hydraulic guidewire having an internal lumen, a distal end and a proximal end; and
an exchange sheath.

2. A system as recited in claim 1, whereby said hydraulic guidewire has one or more apertures at the distal end.

3. A system as recited in claim 2, whereby one aperture is aligned along the longitudinal axis of said guidewire.

4. A system as recited in claim 3, wherein said aperture aligned with said longitudinal axis functions to facilitate the penetration of said guidewire through a total occlusion.

5. A system as recited in claim 2, whereby at least one aperture is directed radially from the longitudinal axis of said guidewire.

6. A system as recited in claim 2, wherein said radially directed apertures function to facilitate the centering of said guidewire within a vessel lumen.

7. A system as recited in claim 2, further comprising an adapter on said proximal end for connecting with a hydraulic means.

8. A system as recited in claim 1, further comprising a removable core that is designed to communicate with said lumen of said guidewire.

9. A system as recited in claim 8, wherein said removable core functions to vary the stiffness of said guidewire.

10. A system as recited in claim 1 wherein said flexible balloon functions to facilitate the centering of said guidewire within a vessel lumen.

11. A hydraulic guidewire comprising:
an elongated hypotube and flexible tubular member forming a composite tubular structure, said composite tubular structure having a proximal end, a distal end, and a lumen extending throughout its entire length;
at least one aperture located at said distal end; and
an adapter with at least one extension mounted on said proximal end of said guidewire.

12. A hydraulic guidewire as recited in claim 11, further comprising a removable core that is designed to communicate with said lumen of said guidewire.

13. A hydraulic guidewire as recited in claim 12, wherein said removable core functions to vary the stiffness of said guidewire.

14. An anchoring, centering and supporting catheter for treating chronic total occlusions comprising:
a multiple coaxial tubular member having a proximal end, a distal end, and at least one lumen extending throughout the length of the tubular member, one first lumen including a distal opening and a proximal opening, whereby one first lumen is designed to receive a guidewire;
a substantially spherical flexible balloon mounted on the distal end; and
wherein said distal balloon functions to substantially center said guidewire within a vessel lumen.

15. An anchoring, centering and support catheter as recited in claim 14, further comprising a series of control wires extending from the proximal end of the catheter to the proximal end of said balloon, wherein said control wires function to align the longitudinal axis of said distal opening with the center of a vessel lumen.

16. An anchoring, centering and supporting catheter as recited in claim 14, further comprising a series of control wires extending from the proximal end of the catheter to the distal end of said balloon, wherein said control wires function to aligned the longitudinal axis of said distal opening with the center of a vessel lumen.

17. A method of treating chronic total occlusions in a patient, comprising the steps of:
advancing a hydraulic guidewire to a location just proximal of a total occlusion treatment site;
advancing an anchoring, centering, and supporting catheter over said hydraulic guidewire;
inflating said balloon on said anchoring, centering, and supporting catheter to a working pressure whereby a proximal opening in said balloon is substantially directed towards the center of a vessel lumen;
advancing said hydraulic guidewire through said total occlusion;
deflating said balloon;
retracting said anchoring, centering, and supporting catheter; and
advancing an exchange sheath over said hydraulic guidewire and through the total occlusion.

18. A method as recited in claim 17 further comprising after inflating said balloon on said anchoring, centering, and supporting catheter to a working pressure the step of actuating a series of control wires to further center said distal opening of said balloon with the vessel lumen.

19. A method as recited in claim 17, further comprising the step of employing hydraulic pressure exiting from radially directed apertures in the hydraulic guidewire to facilitate centering the guidewire while advancing said hydraulic guidewire through the total occlusion.

20. A method as recited in claim 17, further comprising the step of employing a plurality of hydraulic pressure pulses exiting from an aperture aligned along the longitudinal axis of said hydraulic guidewire to facilitate advancing said hydraulic guidewire through the total occlusion.

21. A method as recited in claim 17, further comprising the step of retracting the hydraulic guidewire after the exchange sheath is advanced through the total occlusion.

22. A method as recited in claim 21, further comprising the step of advancing a standard guidewire through the exchange sheath.

23. A method as recited in claim 22, further comprising the step of retracting the exchange sheath.

24. A method as recited in claim 23, further comprising the step of advancing an interventional device over the standard guidewire to the occlusion for additional treatment.
